(19) United States
(12) Patent Application Publication
(10) Pub. No.: US 2006/0173480 A1
(43) Pub. Date: Aug. 3, 2006

(54) Safety penetrating method and apparatus into body cavities, organs, or potential spaces

(76) Inventor: Yi Zhang, Calgary (CA)

Correspondence Address: Robert Platt Bell;Registered Patent Attorney, P.O. Box 310, Aurora, NY, 13026, US

(21) Appl. No.: 11/332,493

(22) Filed: Jan. 13, 2006

Related U.S. Application Data
(60) Provisional application No. 60/647,820, filed on Jan. 31, 2005.

(51) Int. Cl. A61B 17/34

(52) U.S. Cl. 606/185

(57) ABSTRACT

To more accurately control insertion of penetrating instruments (e.g., trocars, needles, or the like) into a body cavity, organ, or potential space, an accelerometer is coupled to the penetrating instrument. The accelerometer may by employed to measure or detect the sudden lack of resistance which occurs when the penetrating instrument penetrates to a predetermined depth (e.g., through the abdominal cavity, vein, or outer bone) in a more accurate and reliable way instead of practitioners' subjective feeling. An acceleration sensor (i.e., accelerometer) coupled to the penetrating instrument (e.g., trocar or the like) may transform the physical variable ‘resistance change’ into an electronic signal that is then processed in an electronic circuit, and finally triggers an audible/visible alarm and/or feeds an actuating mechanism to control movement of the penetrating instrument.

### CROSS-REFERENCE TO RELATED APPLICATIONS

The present application claims priority from U.S. Provisional Patent Application Ser. No. 60/647,820, filed on Jan. 31, 2005, and incorporated herein by reference.

### FIELD OF THE INVENTION

The present invention relates to a medical method and apparatus for penetrating various body cavities, organs and spaces with penetrating instruments such as trocars, cannulas or needles, and more specifically, to an improved penetrating method and apparatus, in which sensors, alarms and/or relative actuation mechanism for stopping the instruments are provided to make an accurate and safe tip placement of penetrating instruments in the body cavities, organs and spaces for adjacent tissue injury prevention.

### BACKGROUND OF THE INVENTION

Many medical procedures, such as minimally invasive surgical and diagnostic procedures and epidural anesthesia, gain access to the inside of body cavities, organs and spaces by using various penetrating instruments for the purposes of observation, treatment, biopsy, and the like. These numerous body cavities, organs and spaces include, various veins and arteries, various hollow and solid organs, bladder, liver, lung, kidney, tonsil, thyroid, cricothyroid membrane, tracheal cartilaginous ring, maxillary sinus, tumor, abscess, pleural and thoracic cavity, peritoneum and abdominal cavity, epidural and subarachnoid spaces, heart ventricles, spinal and synovial cavities, bone ilium and marrow cavity, joint spaces in knees, hips, ankles, discs and shoulders, women's cervical, breast, amniotic cavity, umbilical cord and parts of the fetus, lymph channels and brain ventricles, to mention some of the more common.

There are also numerous types of penetrating instruments specifically designed for the function of every cavity, organ or space penetration, such as various trocars, cannulas or needles. They usually have a sharp or blunt tip with a conical or multi-sided, substantially pyramidal configuration. When they are pushed to penetrate body cavity, organ or space wall with differently required force according to the different type and thickness of the tissue forming the cavity, organ or space wall, they may effectively create small access opening.

When the tip of a penetrating instrument is being pushed through different tissue layers it may encounter relatively different resistances from the tissue layers. The resistance change is determined by tissue type and density. When penetrating a cavity or space wall, it encounters great resistance from the dense wall tissue. As soon as the tip and blade of the instrument pass through the cavity or space wall and into the cavity or space, the resistance drops suddenly and significantly. In these penetration procedures practitioners are generally required to sense the resistance change, especially the sudden lack of resistance, as one of evidences of the correct penetrating tip placement in the penetration process.

For example, the sudden lack of resistance to penetrating instrument is described as ‘give sense’ in epidural anesthesia textbooks. In minimally invasive surgery surgeons describe this sudden lack of resistance as ‘plunge effect’. In an additional example whether the needle is in the marrow cavity may be evidenced by this lack of resistance after the needle passes through the bony cortex. The different descriptions in different specialties and disciplines express the same physical phenomenon. The resistance change or especially sudden lack of resistance may vary significantly between every patient with different sex, age, weight and other anatomic variations of body habitus.

The more experience the practitioner has, the more subtle change he/she may feel. If the practitioner can't feel this lack of resistance and stop the penetrating instrument without delay upon the tip entrance into the cavity, organ or space, the penetrating tip may go too deeply and injure neighboring organ. Even if the practitioners feel the right lack of resistance and try to stop the penetrating instrument immediately upon the tip entrance into the cavity, organ or space, in some cases when the force required in the penetration is great, due to hand movement inertia there is still considerable risk that the instrument may continue penetrating too deeply into the cavity, organ or space and injure neighboring organ.

However careful the practitioner may be during the body cavity, organ or space penetration, there is always a possibility of such danger. In different penetrating procedures, the probabilities of the penetration injuries are more or less in a different degree. For example, a considerable number of the penetration injuries do occur every year in both epidural anesthesia and minimally invasive surgery, in which great attention has been drawn.

So far intensive efforts have been made to solve this fundamental problem of the safe instrument tip placement, but the results are not very satisfactory.

The penetrating instruments may be categorized into two groups. One group has a relatively small diameter and a relatively easy control of the instrument advancement, such as stylet or hypodermic needles. There is no safety mechanism on the instrument and practitioners depend upon experience and/or some add-on process to judge the position of penetrating instruments. The typical is epidural needle for the identification of epidural space in epidural anesthesia. Practitioners usually attach a syringe to the needle and judge the needle entrance into epidural space from syringe injection.

The other group has a relatively large diameter and forces applied onto the instruments may be as high as tens of pounds. Due to much higher injury risk to neighboring organ, this group usually has safety shields. The commonest safety shield is spring-loaded and activated when the instruments enter the cavity or space. The typical is various trocars in minimally invasive surgery.

In both groups, this problem is readily apparent and remains a major determinant of procedure safety. The typical epidural anesthesia, intraosseous infusion and minimally invasive surgery are exemplified to describe this in details.

(1) Epidural anesthesia: In epidural anesthesia, the identification of epidural space is the major procedure determinant. According to authoritative ‘Epidural Anesthesia’ by P. R. Bromage, Philadelphia, W B Saunders 1978, there are mainly four signs to suggest the identification of epidural space: (a) The sudden lack of resistance to advancing needle as it leaves the dense ligamentum flavum to enter epidural space filled loose areolor tissue and vessels, known as ‘give sense’ sign in textbooks; (b) The sudden release of injection of a little air or liquid from a syringe attached to advancing needle, known as Loss of Resistance or LOR method; (c) True and/or potential negative pressure in epidural space, known as ‘hanging-drop’ method; and (d) Vascular and respiratory pressure swings as confirmatory signs.

Presently the commonest method for epidural space identification (ESI) is to attach a LOR (loss of resistance) syringe to the epidural needle and test the sudden release of injection of air or liquid from the syringe upon the entrance of epidural space. The main drawback of this popular technique is mentioned in anesthesia textbooks: two handed needle advancement is not possible. The practitioners have to divide their attention and coordinate their two hands to perform two different functions: to exert and sense gentle pressure on the syringe plunger and simultaneously advance the epidural needle carefully in a millimeter scale. This technique requires performing skills and experience, which are varied and controlled by human factors. It is not a very safe and reliable operation for both novices and experts. Until presently there have always been controversial paper discussions concerning this technique among anesthesiologists for the improvement on operational safety and reliability.

There are also lots of other LOR variations with minor or major changes, some just using the hands with a different grip and others with mechanical aids such as spring-loaded and balloon additions. These mechanical designs may show their advantages in patients with well-defined ligaments, but in others in whom the ligaments have spongy structure and vary in density, slight inward movements of the visual aid (spring may release halfway and balloon may collapse halfway) may cause confusion. For those cases the practitioners have to use their human senses to interpret the position of the needle according to slight changes of resistance in different parts of the ligament. Therefore, the syringe, as the equipment for LOR method has been gaining popularity all the time.

From evaluating major previous reported modifications for ESI, following generalization may be obtained. All of them were based only on either (b) sign or (c) sign, i.e., either the variations of LOR or the variations of hanging-drop. It is noticeable that the more direct and reliable (a) sign has been neglected all the time. Skilled anesthesiologists may usually determine the proper insertion depth for epidural steroid injection (ESI) by feeling this ‘give sense’ of sudden resistance change to needle advancement. So far no single design has been tried to improve the accuracy and reliability of this subjective ‘give sense’ sign sensing. It is also rather noticeable that there is a lack of effort on the combination of these signs, which may be a high possibility of counteracting each sign's drawback and obtaining optimum result.

(2) Intraosseous infusion: When traditional intravenous access is difficult or impossible such as pre-hospital emergency, military, and pediatric patients, one suitable alternative to vascular infusion is intraosseous infusion. U.S. Pat. No. 5,817,052, incorporated herein by reference, describes the technique problems. Bone marrow acts as a non-collapsible vein, through which any drug or fluid can be rapidly and safely administered. Intraosseous infusion requires the penetration by a needle or the like of the patient's skin and outer bone to gain access to the bone marrow. One problem with intraosseous infusion is the practical difficulty of inserting the infusion needle to the proper depth in the bone in order to access the bone marrow. Present techniques can't always provide an effective indicator of the needle's position within the bone, because they use the skin surface as the reference point or because they rely on the user to know the correct anatomical location, and to estimate the required depth. Human subjects show considerable variability in the sizes and thickness of the walls of their bones, of the marrow spaces inside the bones, and of the depth of the layers of skin, muscle, and fat, which make up the tissues overlying the bones. For the above reasons, using the skin surface as a reference point for the practitioners to gauge depth of penetration, and marrow access may be both ineffective due to the low probability of placing the needle in a desired location, or unsafe due to the high probability of placing the needle in a hazardous location such as a tissue compartment, a bone growth plate, a nerve, a great vessel, or the heart.

Another typical approach to the problem of achieving correct placement of an intraosseous system has been to monitor the resistance to penetration of a conventional infusion/aspiration needle. Generally speaking, the resistance is relatively high when the tip of the needle is moving through the outer cortical bone, and it decreases when the tip reaches the marrow space. The resistance increases again if the needle tip reaches the inner cortical bone, on the opposite side of the marrow. However, such variations in resistance may be very subtle and can vary substantially from one patient to another. Further, they require the practitioner to advance the needle very slowly and with considerable skill, often with twisting, in order to not suddenly break through the bone and over-penetrate. Monitoring penetration resistance by human feeling is not considered an effective technique for controlling penetration depth.

Manually inserted needles and techniques, which usually require skill and training for proper use, require a significant amount of operator manipulation during insertion of the needle and necessitate many seconds to minutes in use. An automated needle system would have great utility and better meet the time-value needs for its various pre-hospital and emergency applications.

(3) Minimally invasive surgery: The Prior Art has been discussed in many publications such as U.S. Pat. No. 6,270,484 and No. 5,466,224, both of which are incorporated herein by reference. Traditional surgery was performed using an open technique. The surgeon made an incision dictated by the need to directly observe the area of interest and to insert his or her hand or hands, and/or one or more instruments to perform manipulations within the body cavity accessed through the incision. These incisions may be as long as 20 centimeters, traumatic, painful and may leave unsightly scars. These techniques also require extended prolonged hospitalization and recovery time.

In response to above drawbacks, minimally invasive surgery has been available for over twenty years and has been getting wider and wider applications. Penetrating instruments such as insufflation needle and various trocars are generally the first step to establish endoscopic portals or other relatively smaller incisions for inserting the various manipulative instruments, which are usually 10-25 cm in length and 5-30 mm in diameter. Then there come a number of following procedures, such as laparoscopic procedures in the abdominal cavity, endoluminal, perivisceral, endoscopic, thoracoscopic, intra-articular and hybrid approaches. For example, the laparoscopic procedure may be used in performing cholecystectomy, appendectomy, herniorrhaphy, hysterectomy, vagotomy pericardiotomy, esophagectomy, oophorectomy, gastral and bowel resections, nephrectomy, and the like.

Since the diameters of the trocars used are relatively larger in the range of millimeters (much lager than the epidural needles) and more force, typically ranging from several to tens of pounds, is needed to push the trocar for the penetration. Therefore, the risk of internal organ injury is greater and trocars thus usually have safety features.

Trocar designs with various safety features generally fall into protruding and retracting categories, or combinations of protruding and retracting categories. In protruding safety trocar designs, a safety member is spring biased to protrude beyond the trocar tip in response to the reduced force on the distal end of the safety member upon entry into the cavity, organ or space. The safety member may be disposed around the penetrating member in which case the safety member is frequently referred to as a shield, or the safety member may be disposed within the penetrating member in which case the safety member is frequently referred to as a probe. The force required for penetrating the cavity, organ or space wall necessarily includes the force required to overcome the spring bias on the safety member as well as the resistance of the cavity wall. To enable the safety member to protrude after penetration, the spring bias on the safety member and, consequently, the force to penetrate the cavity, organ or space wall have to be increased considerably. It also increases the difficulty in trocar control in the penetration.

In retracting safety trocar designs, the penetrating member is retracted into the cannula upon entry into the cavity, organ or space, in response to distal movement of a component of the safety trocar such as the penetrating member, the cannula, a probe or a safety member such as a shield or probe. These trocars have the disadvantages of requiring relatively complex mechanisms to hold the penetrating member in an extended position during penetration and to release the penetrating member for retraction and, concomitantly, not retracting sufficiently quickly and reliably.

In safety trocar designs that combine elements of the protruding and retracting instruments, typically, the penetrating member of the safety trocar is retracted and one or more safety members are extended to protrude distally beyond the distal end of the penetrating member. These trocar designs are a compromise and unable to overcome the inherent disadvantages.

In fact, from the view of reliability engineering of instrument design, the simpler the safety mechanism, the more reliable the trocar may be. While the more complicated trocar design with more components may provide better functional safety, the practical reliability of the whole trocar consisting of more components is reduced as a compromise.

Many factors including anatomic variation of different body habitus and practitioner's human uncertainty influence and complicate the situation and a more sophisticated apparatus or technique is needed. For example, most delicate organs are very close to the inside of the skin layer being penetrated, while the filled carbon dioxide puts them apart to increase safety margin, the force required for penetration and the elastic nature of the muscular layer cause a severe depression at the penetration site, therefore bringing them closer to counteract the insufflation effect. Furthermore, the friction between tissue wall and safety shield retards the deployment of the safety shield.

To establish the entry of the peritoneal cavity is first and major risky step for all procedures. There are basically two kinds of techniques. The first includes direct vision techniques including the typical optical trocar technique. The other is the classical blind technique typically with insufflation needle insertion. Organ injuries have been reported with all techniques and none of them satisfy the clinical need while each of them is practiced in present minimally invasive surgery.

There are a variety of trocar forms for penetration safety: shielded pyramidal, shielded blade, conical, radial expandable, optical, winged cone, short-stroke knife, Veress needle, Hasson open/blunt trocar. No particular apparatus has been illustrated to be safer (Journal of the American College of Surgeons. 2001 April;192(4):478-90; discussion 490-1, and ‘Trocar injuries in laparoscopic surgery,’ Journal of the American College of Surgeons 2001 June;192(6):677-83, both of which are incorporated herein by reference). Among them even the Hasson-type, open-incision, blunt cannulas are associated with small bowel injury, which might be lethal, retroperitoneal vascular injury, death, as well as abdominal-wall vessel laceration, and other visceral injury (Journal of the American College of Surgeons 192(4) April 2001, incorporated herein by reference).

Clinical results have illustrated that the safety features of all existing trocars are incapable of truly effective prevention of injuries in penetration and need substantial improvement.

Most complications in minimally invasive surgery occur at the time of insufflation needle and trocar insertion. Among all the contributing factors, the feel of tissue resistance and the force control of trocar insertion are very crucial. Since trocar malfunction is rare and most injuries involved devices that appear to be functionally normal, this persistent hazard calls for an urgent need to strengthen the continuing search for substantial improvement in trocar insertion techniques.

Minimally invasive surgery is expanding to more and more applications in many specialties and presents an opportunity to improve overall surgical procedures. However, the persistent problem of trocar insertions has hindered the development. In 1996, the FDA Center for Devices and Radiological Health addressed the problem of shielded entry trocars and its 2003 annual report pointed out again “the increasing numbers of reports of deaths and serious injuries related to the use of laparoscopic trocars”.

FIG. 2 is a simplified diagram illustrating the penetration process of the Prior Art for a cavity, organ or space wall. In stage A, the tip of the penetrating instrument 210 is in the dense wall 220 with great resistance. In stage B, the tip just pierces the wall and there may be sudden resistance change at the puncture moment. In stage C, the tip and portion of the cutting edge are in the cavity or space. In stage D, the tip and all the cutting edge are in the cavity or space. In stage E, a certain length of instrument cylinder body is in the cavity or space.

Because Prior Art safety feature deployment devices need a penetrating hole, the penetrating depth of their protection is often after the critical stage D, and somewhere more or less in the stage E, which is within the dangerous depth. In addition, the time lag of safety feature deployment, and friction between wall tissues and the mechanism retard the effectiveness of the real protection. These are theoretical reasons of inevitable injuries that clinical results have demonstrated for all the past years.

In existing safety trocars, the force required to penetrate the cavity, organ or space wall includes not only the force required to pass the safety-penetrating instrument through the wall but also the force required to overcome the spring bias on the safety shield. To overcome the friction with the surrounding wall tissues and assure protective distal movement of safety shield effectively upon the entrance, the strength of the spring biasing the safety shield may be sufficiently increased. However, increasing the strength of the bias spring also increases the total force required to push the penetrating instrument and results in more difficulty in controlling the instrument movement.

Accordingly, all existing safety trocars have been designed to compromise force-to-penetrate and assured safety shield movement in an attempt to satisfy both requirements.

### SUMMARY OF THE INVENTION

Various embodiments of the present invention guarantee that the penetrating tip enters the critical stage D in a safer and more cautious way other than in a rush and uncontrollable way. The deeper penetration beyond stage D may be controlled within a small estimated increment as accurate as millimeter scale other than an unknown and uncontrollable depth in all existing safety trocars. Thus, the danger of adjacent tissue or organ injuries may be greatly reduced. In addition, the present invention remains (and does not change any) original safety features of all trocars, but rather, provides an additional safety feature. From this point, the penetration safety may be guaranteed by two independent safety systems and the penetration danger be doubly reduced. Even furthermore, because the present invention enables the penetrating tip to enter the dangerous cavity or space region in the shallowest and controlled depth, in the event of an injury, it may be a slight contact injury, as opposed to a serious cutting or stabbing injury.

With the controllable movement of penetrating instrument under the present invention's add-on safety protection, existing safety trocars may not have to compromise the two requirements and may be redesigned to increase the strength of the bias spring further to assure the protective effectiveness of the safety shield. Thus, in combination with the add-on safety feature of the present invention, the original safety effectiveness of existing trocars may be improved definitely.

As it is aforementioned, one form of trocar system has sharp pyramidal cutting edges. The sharper the edge, the less force is required for insertion. Another form of conical trocars tends to cause less wound bleeding because they are non-cutting. The non-cutting trocars are superior because of reduced wound complications, but there is no apparent improvement in damage to either deep vessels or viscera with the conical trocars. This may be related to the additional force required to insert conical trocars. The additional force needed for penetration allows for less control of the cavity or space entrance and the injury probability may be increased with difficult trocar insertions.

The add-on safety feature of the present invention detects penetration at the earliest time by means of an electronic acceleration sensor, instead of practitioners' subjective feeling with delayed perception time, and thus provides a very effective non-interfering force control of trocar insertion. This add-on feature may let both the trocar design and relative safety shield design have less compromise and superior advantages. Thus, the overall trocar safety may benefit in a comprehensive way.

During penetration procedures through two different tissue layers or cavity and space wall, the physical phenomenon of resistance change or sudden lack of resistance should be described more accurately and scientifically. In the concept of the present invention, it may be described more accurately and scientifically as acceleration. Therefore, an acceleration sensor may be used in the present invention to measure the degree of the resistance change, and a subtle resistance change that may not be discerned by practitioners' sense may be detected reliably.

As one of the most fundamental principles of Physics, Newton's second law (a=F/m) asserts that the net force acting on an object gives the object an acceleration, which describes the rate of the object's velocity change. When the penetrating instrument is in the dense cavity or space wall, the resistance to the penetrating instrument and the pushing force acting on the instrument are approximately in balance (the latter may be a little greater). Upon the entrance of cavity or space, the sudden lack of resistance to penetrating instrument causes an unbalanced force and the instrument has a forward instant acceleration. In fact the practitioner may feel this instant acceleration as a ‘give sense’ or ‘plunge effect’. It should be understandable that above phenomenon is a typical example that Newton's second law describes exactly.

This acceleration expression of resistance change in various penetration procedures has not been found in all previous literatures including classical textbooks and modern Patents. The present invention is the first to put up this concept and all the designs of the present invention are based upon it. In comparison with all other patents, the present invention has tackled the intrinsic part of the anatomical structure in penetration procedures and opened a brand new logical direction for future improvement attempts.

### BRIEF DESCRIPTION OF THE DRAWINGS

FIG. 1 is a block diagram illustrating the major components of the preferred embodiment of the present invention.

FIG. 2 is a simplified diagram illustrating the penetration process of the Prior Art.

FIG. 3 is a block diagram illustrating the major components and signals for an embodiment for performing an epidural procedure.

FIG. 4A is an exploded and cross-sectional view illustrating one embodiment of a disposable dual-sensor configuration of the present invention.

FIG. 4B is an exploded and cross-sectional view illustrating another embodiment of a disposable dual-sensor configuration of the present invention.

FIG. 5A is an electrical schematic diagram illustrating a low cost circuit embodiment of one embodiment of the present invention.

FIG. 5B is an electrical schematic diagram illustrating a low cost circuit embodiment of another embodiment of the present invention.

FIG. 6 is a side perspective view illustrating the restraint mechanism of one embodiment of the present invention.

FIG. 7 is a side elevational view of the restraint mechanism of FIG. 6.

FIG. 8 is a block diagram illustrating the major components of the restraint mechanism of the present invention.

### DETAILED DESCRIPTION OF THE INVENTION

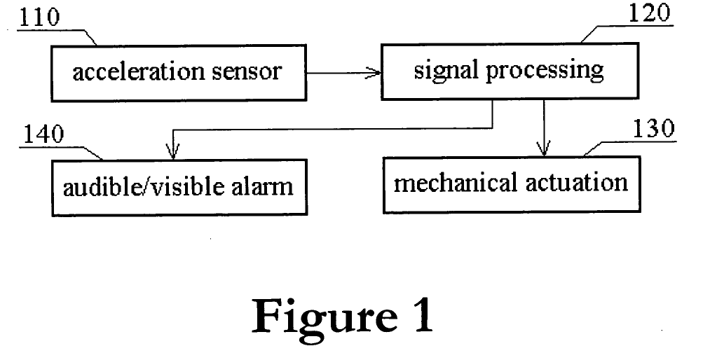

FIG. 1 is a block diagram illustrating the major components of the preferred embodiment of the present invention. In recognition of acceleration expression, an acceleration sensor may by employed to measure or just detect this sudden lack of resistance sign in a more accurate and reliable way instead of practitioners' subjective feeling. The basic elements of the present invention are illustrated in FIG. 1, which is understandable to people with general electronics background. An acceleration sensor (i.e., accelerometer) 110 coupled to the penetrating instrument (e.g., trocar or the like) may transform the physical variable ‘resistance change’ into an electronic signal that is then processed in the electronic circuits 120, and finally triggers an audible/visible alarm 140 and/or feeds an actuating mechanism 130 to control movement of the penetrating instrument.

There are many types of penetrating instruments for different specialty applications. To apply the present invention concept in all existing penetrating instruments, the specific product designs may be in three different forms to meet the needs of specific instruments, although they may share the same electronic principle diagram in FIG. 1.

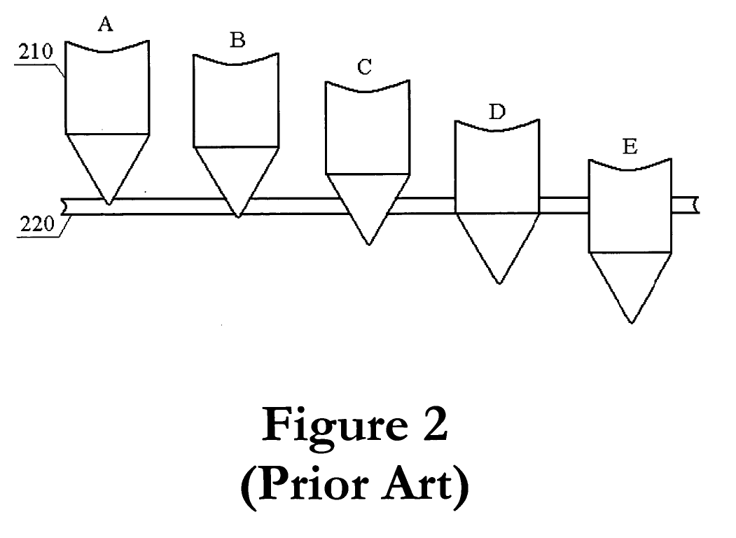

The apparatus of FIG. 1 may be provided as a disposable miniaturized model. It may provide an audible and/or visible alarm at the earliest penetration stage B of FIG. 2, and leave a longer time period for practitioners to stop instrument movement. It may be designed so tiny as to not influence penetration movement adversely. It is an add-on safety apparatus and does not have to change any part of existing instruments. The apparatus may be conveniently worn at the practitioners' fingers or wrists, or attached on penetrating instruments by means of various mechanical connections, such as hub attachment or mechanical clamp. This miniature design may be suitable for all types of penetrating instruments, and especially more suitable for penetrating instruments with relatively small diameters and blunt tips.

In another embodiment, a restraint apparatus may be provided. The electromechanical apparatus is designed to have both electronic monitoring/alarming circuits and mechanical actuating mechanism to control or restrain penetrating instrument movements or practitioners' hand movements. Because its usage is either not in contact with the penetrating instrument or alternatively, easily detachable from the proximal part of a penetrating instrument, it is an add-on safety apparatus and does not have to change any part of penetrating instruments. Thus, the present invention may be applied to an existing instrument without having to redesign the basic instrument.

With the earliest acceleration feedback of penetration, it may force practitioners' hands and holding penetrating instrument to move under mechanical control limitation, to only make small, predetermined increments and eradicate excessive movements and human errors, and to ensure that the penetrating tip enters the dangerous cavity or space region in the shallowest, estimated and controlled depth. It is suitable for all penetrating instruments in principle and practically may be a necessity in some more risky applications.

In another embodiment, the presenting invention may be integrated into a surgical instrument. The apparatus of the present invention may be incorporated into specific penetrating instrument body. Because the acceleration sensor does not need to be disposed at the distal end of trocars (usually at proximal hand-holding part of trocars conveniently away from tip and long cylinder body of trocars), it may facilitate the mechanical design of safety shield on trocars. With the earliest and reliable acceleration feedback of the penetration, the penetrating instrument may be redesigned to improve existing safety members, and add a new safety mechanism with acceleration feedback, and finally become a fully automated penetration instrument.

As aforementioned, the penetrating instruments may be categorized into two groups: with or without safety features. The epidural anesthesia and minimally invasive surgery applications are typical examples for two groups. The specific designs for all penetrating instrument in other applications may refer to these examples, but should not be limited to them. All three design forms may increase safety margin with more or less degree to any specific penetrating instrument. It may be determined by careful consideration and clinical tests that which of the three is the best suitable with most effectiveness.

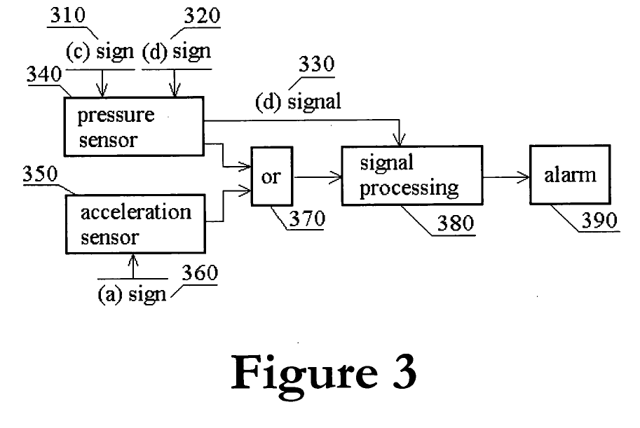

FIG. 3 is a block diagram illustrating the major components and signals for an embodiment for performing an epidural procedure. In the application of epidural anesthesia, on the basis of aforementioned ESI analysis, the optimal idea of the present invention for ESI has been formed to utilize all the possible advantages and avoid relative disadvantages. The new design may be based on (a) sign 360 and (c) sign 310, as well as (d) confirmatory sign 320 in a multi-sign integration. To avoid false positive signs, the threshold value for (c) sign 310 of negative pressure is set higher than that of (d) sign 320 of pressure swing (usually much smaller).

The (a) sign 360 and (c) sign 310 may have an OR relation 370, that is, either of the signs may give out an ESI alarming signal 390, as illustrated in FIG. 3. The (a) sign 360 may be utilized alone to give out this first ESI alarming signal 390. In another embodiment, the (a) sign 360 may have an AND relation (not shown) with a new pressure sign (its value between the first alarm pressure sign and pressure swing sign) and then give out the first ESI alarming signal 390. With or without the first alarm for ESI, the (d) sign 320 may be checked and give out another synchronous alarm from signal 330 for pressure swing presence to confirm the ESI. Thus, the design utilizes all the available signs to provide an easier operation procedure with higher safety, reliability and convenience.

As illustrated in FIG. 3, the electronic device comprises an acceleration sensor 350 to measure or detect (a) sign 360 of loss of resistance, a pressure sensor 340 to measure (c) sign 310 of negative pressure and (d) sign 320 of the confirmatory pressure swings, related sensor signal processing circuit 380, and an alarm unit 390. The sensors 340, 350 may transform physical variables such as acceleration and pressure, into electronic signals, which may be combined in OR relation 370, or directly sent (e.g., (d) signal 330) to signal processor 380, where they are then processed, to finally trigger the alarm unit 390.

The operation procedure of this automated device is similar and simpler to that of a LOR syringe. When the epidural needle is in the ligament, instead of attaching a LOR syringe, the syringe-shaped device is attached to the needle hub. The practitioner may then use their two hands and concentrate all attention to careful needle advancement, in contrast to two-hand coordination to perform two different functions and attention division between keeping/sensing pressure on the plunger and needle advancement with LOR method. Upon the entrance of epidural space, a first alarm may be automatically generated for pressure and/or resistance change detection and then a second respiration synchronous confirmatory alarm to ensure the correct needle position. According to Bromage's textbook, patients may be instructed to have some deep respirations (or coughs), and this patient cooperation may increase pressure swings and ensure the confirmation sign further.

For low cost disposable use, the piezoelectric materials such as polyvinylidene fluoride known as PVDF and ceramics may be employed for sensor design in consideration of its directly driving CMOS capability. A CMOS version of timer chip 555 may employed as a subsequent monostable circuit for signal detection and alarm of the above dual-sensor. While piezoelectric ceramics are a better candidate for their lower cost, PVDF films have much higher length voltage coefficient g₃₁ and are more suitable for this application of tiny signal detections.

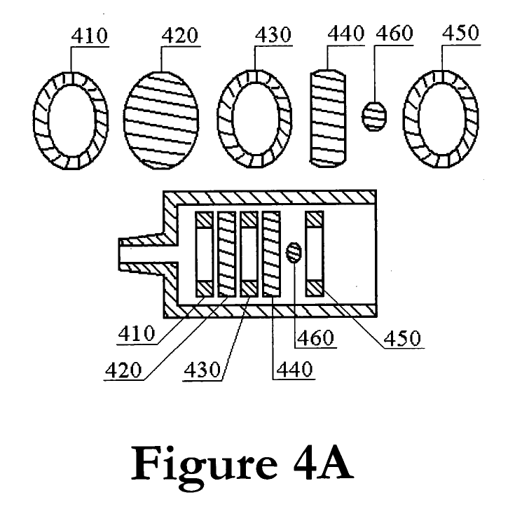

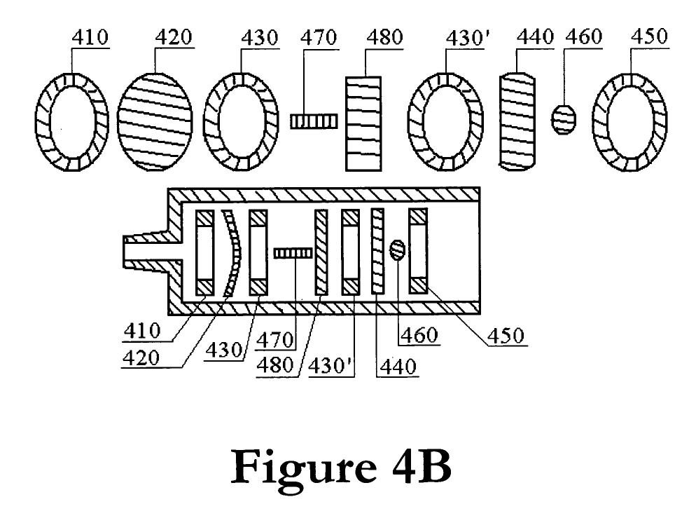

As illustrated in FIG. 4A, a piece of round PVDF film 420 may be sandwiched between two ring-shaped fixing frames such as ring gaskets 410 and 430, by mechanical means or adhesives, to form a differential pressure detecting element. When there is a pressure difference between two surfaces of PVDF film 420, that is to say, if one surface of film 420 is in atmospheric pressure, and there is a pressure below atmosphere on the other surface of film 420, film 420 may produce electrical charges, (i.e., electrical voltage) across two surfaces. PVDF film strap 440 is clamped in two ring gaskets 430 and 450, in a similar way to form an acceleration detection element. A seismic mass 460 is glued to one surface of PVDF film 440 as in conventional acceleration sensor design.

An electrical voltage may be present between the film two surfaces, when an acceleration stimulus, such as sudden lack of resistance of the element, applies to the element. In such a structure, ring gasket 430 may have a small thickness to isolate the pressure and acceleration elements. The pressure sensing part in the sandwich structure of gasket 410, film 420 and gasket 430 has its own electrode wire which may electronically conduct with the film surface, and lead to subsequent electronic circuit. The acceleration sensing part in the sandwich structure of gasket 430, film 440 and gasket 450 also has its own electrode wire which may electronically conduct with the film surface and lead to subsequent electronic circuit. If ring gasket 430 is made from electrically conducting material such as metal, it may be used as a common electrode to lead the sensor signal to directly form an OR relation of pressure and acceleration outputs through only one wire in connection with subsequent circuit. For piezoelectric ceramics the sensor technique in FIG. 4A may be simplified: instead of all ring gaskets, adhesives such as epoxy resin may serve the functions well. As an alternative, the two sensing parts of pressure and acceleration detection may not necessarily be incorporated together and the acceleration sensing part may be mounted on PCB with other circuit components.

In the above sensor structure, when there is a sudden (dynamic) negative or positive pressure, the pressure sensing part of PVDF film may give out the same polarity output with the same sensitivity according the theoretical working principle of piezoelectric films. However, the contact area between ring gasket 410 and PVDF film 420 can be increased if the hole of ring gasket 410 is designed smaller. The increased contact area with a smaller inner diameter of ring gasket 410 may counteract some force that the negative pressure causes on the film and the negative pressure sensitivity may be reduced lower. Thus, the pressure sensing part in FIG. 4A may be designed to have a higher sensitivity for positive pressure detection and a lower sensitivity for negative pressure detection. If the ring gasket 410 is made of a net, the sensitivity difference may be bigger. In this choice, the pressure swing detection may utilize the positive pressure detection at a higher sensitivity, because either negative or positive pressure detection is acceptable for pressure swing detection due to deep respiration. The negative pressure detection at a lower sensitivity may be utilized for the negative pressure detection of the first alarm and/or for other signals for reducing possible false first alarms.

FIG. 4B illustrates another embodiment of the device of FIG. 4A. For higher operating sensitivity and lower cost, using a smaller piece of piezoelectric film, part 420 may be provided as an elastic film such as thin silicon rubber film instead of piezoelectric film. Only a narrow strap 470 in the stretch direction of PVDF film may be used in this configuration. One tip of the piezoelectric PVDF film strap 470 is glued onto the center of elastic film 420, and the other tip of PVDF strap 470 is fixed onto the center of a beam 480, which is fixed on the cylinder housing in the diameter position. Thus, elastic film 420 and beam 480 are in parallel position and between them piezoelectric film strap 470 is perpendicular to both of them.

The distance between elastic film 420 and beam 480 may be adjusted to a suitable length, in order that piezoelectric film strap 470 is pre-tensioned properly and the elastic film 420 is in a slight bulged shape. In this configuration, elastic film 420 may be used to transmit the dynamic pressure (force) onto piezoelectric film strap 470, which works at a higher operating sensitivity for dynamic pressure detection in the film stretch direction. When there is a dynamic negative pressure, elastic film 420 may increase tension on piezoelectric film strap 470. When there is a dynamic positive pressure, elastic film 420 may reduce tension of piezoelectric film strap 470. Thus, the signal polarity for negative and positive pressure detection is opposite, which is different from that of FIG. 4A.

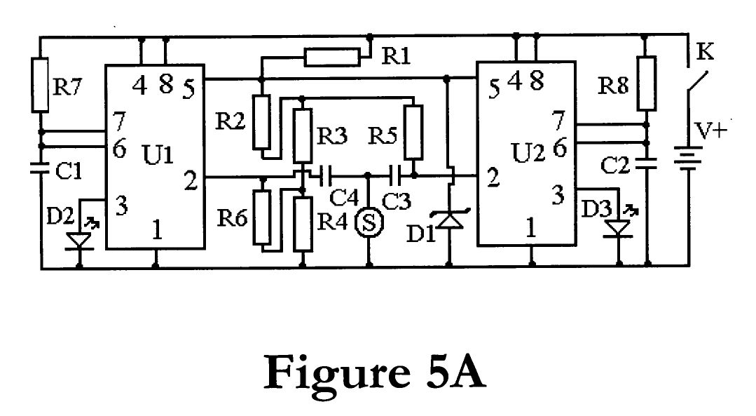

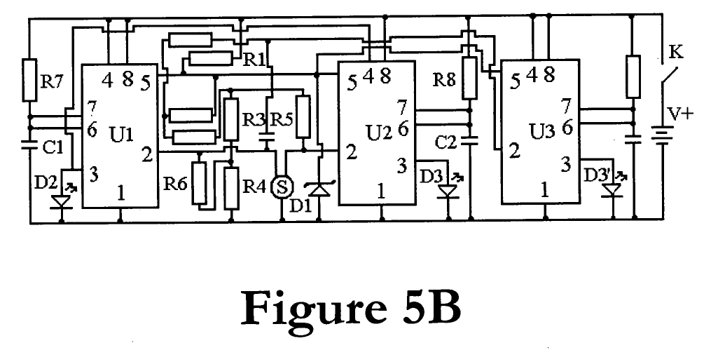

For a sensor structure with OR relation of pressure and acceleration signals, the subsequent detection circuit is illustrated in FIG. 5A. U1 and U2 are both standard CMOS timer chips. The monostable trigger with U2 is for sensor acceleration or pressure signal detection. The monostable trigger with U1 is set with a lower trigger threshold and for the smaller pressure swing detection. Both trigger circuits operate independently to light different color LED alarms D2 and D3. The sensor signal is directly connected to trigger terminal 2, because the CMOS timer chips may be directly driven by a piezoelectric signal.

Diode LM 385 D1 may be used to provide voltage reference for signal comparator design. Resistors R2, R3 and R4 form a divider network and are used to determine the voltage applied to the trigger terminal 2, which is a little higher than half of the voltage of terminal 5 defined by the diode LM 385 D1. The ratio of (R3+R4)/(R2+R3+R4) determines the sensitivity of the acceleration and pressure detection and the ratio of R4/(R2+R3+R4) determines the sensitivity of the pressure swing detection. The large resistors R5 and R6 may be in the range of 10⁸ to 10⁹ Ohms to limit the low frequency response of sensor signals.

In FIG. 5A, a monostable trigger U1 with a lower trigger threshold for smaller pressure swing detection may also be triggered by an acceleration signal. The design works on the condition that there is no acceleration signal to disturb the synchronous output for pressure swing due to deep respiration, when practitioners stop needle advancement and observe the presence of pressure swing. During needle advancement, the U1 output will not be observed because its more sensitive pressure and acceleration output may give too many signals without meaningful use for the ESI.

For a sensor structure with separate acceleration and pressure output wires, the monostable trigger U1 and U2 work independently to light different color LED alarm D2 of pressure swing signal and LED alarm D3 of only acceleration signal, respectively. Without the OR relation of pressure and acceleration signals for the first alarm, the acceleration sensitivity may be set higher to ensure high occurrence of first alarm. Because of some spongy nature of ligament or other anatomical reasons, there is a possibility of false first D3 alarm for ESI by any acceleration detection. Although the second D2 alarm of pressure swing due to deep respiration may distinguish it, it may be better to have less false first D3 alarms for ESI if there are any. According to dura tenting theory of epidural space, epidural space has both the sign of tiny generated negative pressure and the sign of resistance change, which may be different from the possible resistance disturbance before epidural space entrance.

In consideration of this point, the reset terminal 4 of U2 may be disconnected from high voltage V++, and connected to output terminal 3 of U1, as shown in FIG. 5B. FIG. 5B is in accordance with FIG. 4A with the same signal polarity for negative and positive pressure detection to illustrate this variation. For the sensor configuration in FIG. 4B with opposite signal polarity for negative and positive pressure detection, the sensor wires may be selected from two respective PVDF film surfaces for negative and positive pressure detection and make FIG. 5B suitable as well. In practical circuit design for simple sensor wiring of FIG. 4B, FIG. 5B may have other obvious variations, in which the trigger units may also select positive pulse as input signal and work in a combined basic monostable modes. For the detection of a sudden negative pressure, the trigger U1 may detect it at a lower sensitivity, which may be designed as a suitable value somewhere between the detection of pressure swing and the detection of threshold pressure sign for first alarm. Thus, the triggering condition of the first alarm may be changed as follow: occurrences of both acceleration and the suitable pressure signs. This simple variation may reduce possible false first alarms for ESI if there are any.

If a third trigger unit is included in the design, it may be used to realize the OR relation of pressure and acceleration signals instead of sensor structure simplification. As illustrated in FIG. 5B, this third unit can independently detect the negative pressure threshold sign for the first alarm and light an additional LED D3′ with the same color of LED D3. Thus, the additional unit and unit U2 make the OR relation of pressure and acceleration signals and the triggering conditions of the first alarm may be as follows: (1) occurrences of only acceleration sign, or (2) threshold pressure sign. However, the advantage of this alternative circuit with OR relation function is that the independent pressure swing detection will not be disturbed by any acceleration signal. Similarly, reset terminal 4 of U2 may be disconnected from high voltage V++, and connected to output terminal 3 of U1. Thus, the triggering conditions of the first alarm for ESI may be changed as follows: (1) occurrences of both acceleration and the suitable pressure signs, or (2) threshold pressure sign.

The above sensor and circuit embodiments, as well as other possible variations are provided to analyze various problems and try to provide relative solutions for best clinical results. While some of them may be a little over-engineering, others may make different versions of practical product design for different practitioners' preferences, all within the spirit and scope of the present invention.

The entire CMOS type circuit device may be supplied power through the use of small button batteries. The syringe-shaped housing for the sensor and electronic circuit may be made of low cost plastic material and a layer of electronically conducting film such as low cost metal films may be coated on the housing for electronic shielding.

The dominating LOR technique has illustrated that new technical advances in ESI are needed to address its three problem areas: two-handed needle advancement; prevention of accidental dural puncture; and false positive sign distinction. The embodiments of this invention are superior to present LOR syringe technique in the solution of all these problems.

Two-handed needle advancement: An alternative intermittent LOR technique may be preferred by some anesthesiologists, especially when the ligaments are difficult to penetrate or a blunter needle is preferred. Two-handed advancement of the needle makes the penetration in an easier and more accurately controlled way, but the penetration has to be stopped and the resistance to injection has to be tested after each advancement, on a millimeter scale. The drawback of this alternative is higher possibility of dura puncture between each actual advancement. The technique of the present invention allows two-handed needle grip and advancement without any compromise and enables practitioners to concentrate all their attention in careful millimeter needle advancement without syringe plunger pressing effort.

Prevention of accidental dural puncture: As quoted from authoritative epidural textbook by P. R. Bromage, less than 0.5 percent accidental dural puncture may be reasonable data, and 1 percent is acceptable from a practical viewpoint. Unfortunately, the actual incidence of dural puncture is often higher than this, particularly in the hands of novices. In the embodiments of the present invention, this failure rate may be reduced approximately tenfold from that of the above data.

The ligamentum flavum is composed of tough elastic fibers in most cases. However, there are some cases that it has a spongy nature and varies in density. Under such circumstances LOR method cannot be demonstrated because the air or liquid in the syringe may escape from the porous structure and cannot be kept in a pressurized state. However, this porous structure may still have pores tiny enough to provide resistance to the blunt tip of advancing epidural needle and give acceleration sign upon entrance of epidural space. The acceleration sign may be lost in a more porous structure with relative bigger pores comparable to the needle tip. From this point the acceleration sign may have a higher occurrence rate than that of LOR, although there is no direct comparable clinical data between them. The puncture process clarifies that lack of resistance to advancing needle is the direct intrinsic process sign, and LOR injection is the artificial sign of another add-on process. The former may be more dependable.

According to the widely accepted Cone theory, the tenting of dura causes negative pressure in epidural space, although there exists real existing negative pressures in some puncture positions. The creation of negative pressure by dura tenting is usually small and sometimes cannot be observed by the hanging-drop method. A more sensitive detection of negative pressure may have a higher occurrence rate than the reported 88% of the hanging-drop. In addition, with a more sensitive detection of negative pressure the dura may be less tented to give the sign and there may be a more margin for dura safety.

Even in the most unfavorable estimation, the occurrence rate of (c) sign is 88% and the occurrence rate of (a) sign is equal to that of LOR. By rewriting the equation, the failure rate of (a) sign is equal to that of LOR. According to multiplication law for independent events of probability theory the failure rate of the design (without both (a) and (c) signs) is equal to 0.12×failure rate of (a) sign, or 0.12×failure rate of LOR at least. As we know, the ESI is the major controlling factor of accidental dural punctures and its failure rate may be regarded as that of accidental dural punctures. On this assumption, the failure rate of ESI, or the chance for accidental dural punctures, may approximately be reduced tenfold.

A report in Chinese Journal of Anesthesia 1983, vol. 3, No.1, p42-43, (incorporated herein by reference) investigated 964 cases from 1978.10-1981.8 in a county hospital. The occurrence rate of negative pressure is 90.87% and that for lack of resistance sign is 97.10%. No case was without either of the two signs. In other words, there is at least one of (a) resistance lack sign and (c) negative pressure sign in all 964 cases. Even though the more sensitive electronic detection instead of human feeling is not considered, the device of the present invention may provide the first audio signal at 0.1% failure rate level. However, according to calculation based on occurrence rates, the device failure rate for the first ESI signal is 0.27%. The data variation may arise from limited cases and further study with more cases may give out a more real probability data. In consideration of the data from both novice and skilled practitioners in the hospital, the data is an encouraging clinical support to the analysis of the present invention.

False positive sign distinction: LOR technique has the problem of false positive results, which have a sign occurrence when the needle is not in epidural space. It may be because of the spongy nature of the ligament, because the point of the needle has entered a small cyst, or because the point has wandered too laterally into the yielding tissue of the erector spinae. Anesthesiologists may be perplexed by these phenomena in occasional cases. The technique of the present invention provides possible term of occurrences of both acceleration and suitable pressure signs, additional confirmatory signs of vascular and respiratory pressure swings to distinguish them.

The hanging drop method observes the inward movement of liquid on the needle hub. The movement needs to overcome the friction on the inner needle wall and has a large sensitivity limit. Therefore, it may not be able to detect some tiny pressures, which cannot move the liquid because of friction. In fact, the detection of tiny pressures seems useful, because there are some useful tiny pressure information such as vascular and respiratory pressure swings which may make additional confirmatory signs for ESI. According to textbooks and clinical reports, the hanging drop method is able to observe most or some (different from clinical tests) of these swing signs and have determined them as useful confirmatory signs, especially in some perplexed cases. With a higher pressure sensitivity these confirmatory signs may have a higher occurrence rate and reliability. Thus, a more sensitive and reliable detection of tiny pressure instead of the hanging-drop is very helpful and a suitable pressure sensor may be the best candidate.

The hanging-drop method may have confirmatory signs of pressure swings to distinguish false positive results. Since hanging-drop is unreliable and the LOR is the most popular, this confirmation sign affiliated to the hanging-drop has not been properly utilized. Instead of one detection sensitivity for both pressure and smaller pressure swing detection in the hanging-drop method, the embodiments are designed to have two different detection sensitivities (different alarm thresholds) for bigger pressure detection and smaller pressure swing detection, the confirmatory sign may give an even better result for the correct ESI.

Other benefits: A recent study from Anesthesia 2002, 57, p768-772, incorporated herein by reference, showed that in all cases the ESI by means of pressure change occurred fractionally earlier than the ESI by LOR. This time superiority is very crucial for the practitioner to have more time to halt the needle on millimeter scale advancement. In the study a pressure sensor was utilized instead of the hanging-drop and took no time to give out an audio signal, but the human sense of resistance to syringe injection consumed perception time. As it is well known that the perception data in driving knowledge ¾ to 1 second is as a reference, this factor is obviously important to leave more time for practitioners' reaction and more space in operation safety margin.

Some anesthesiologists prefer a sharper point needle end instead of a rounded blunt one to push the needle through the ligamentum flavum, which is difficult to penetrate in some cases. It is more likely to puncture the dura. In the embodiments of the present invention, two-handed needle grip and advancement make the penetration easier and a sharper point needle is not necessary. Thus, it may be less likely to puncture the dura for these anesthesiologists.

From the point of clinical convenience, anesthesiologists don't have to go through the procedures of filling the syringe with fluid, which is associated with both LOR and hanging-drop techniques. Instead they only need to press a button to switch on the device and then concentrate all their attention to needle advancement and are then reminded by the ESI signals. Thus, the operation procedures have actually been simplified and shortened for the practitioners' convenience.

One major LOR advantage is that the fluid injection ahead of the needle pushes the dura away from the needle tip, thus reducing the possibility of dural puncture. However this advantage seems to be limited, since its actual incidence of dural puncture is still very unsatisfactory, as aforementioned. Although the embodiments of the present invention may not have this pushing-away advantage, it has other combined advantages of much higher success rate, less dura tenting, less perception time for earlier needle halt, safer two-handed needle grip and advancement. All the superiority of the embodiments of the present invention may exceed and give a better final result.

Superiority to Other Modifications of ESI Techniques: From above analysis, the present invention is the first in the world to utilize the most reliable sign of resistance lack to advancing needle by means of acceleration detection, to combine it with the sign of negative pressure as well as the confirmatory sign of vascular and respiratory pressure swing. It is completely different from all other ESI techniques in both concept and function, which are just modifications of hanging drop or LOR methods.

As mentioned above, mechanical aids such as spring-loaded and balloon indicators rely simply on a predetermined mechanical force and are unable to work with some confusing cases because of extensive patient variations such as ones with spongy ligaments, although they provide the advantage of two-handed needle advancement. In addition, they showed no better or even worse results of accidental dural punctures and had no capability of false positive sign distinction. The embodiments of the present invention improve not only the essential part, but also almost every aspect of ESI clinical results.

In conclusion, from all above analyses in comparison with LOR technique, the embodiments of the present invention have the following features: The success rate may be increased tenfold; Two-handed needle grip ensures more accurate millimeter controls; Possibly limiting false positive results; Additional confirmatory sign; Reduced human errors by electronic detection; A bigger combined margin of safety; Access to onlookers for training and supervision; Less in size and weight as well as simpler operation procedures; and Operation in a more confident and relaxed way.

The embodiments may be modified to be in the form of disposable miniature device for other applications. In FIGS. 4A and 4B of the sensor configuration, elements 410 and 420 as well as 470, and 480 may be removed for just acceleration detection. In FIGS. 5A and 5B the relative U1 and U3 parts may also be removed. Thus, the simplified design may detect acceleration representative of resistance change in various penetration processes by an alarm.

In the application of intraosseous infusion, an acceleration sensor may be employed to detect penetration resistance changes and actuate an integrated electromagnet friction brake to stop needle advancement automatically. The required skin piercing force may be much less than the minimal penetration force onto the bone and there is a resistance increase. This is the first landmark of negative acceleration value, which may make a more accurate needle depth reference from bone surface instead of skin surface reference. In the further needle advancement, upon marrow entrance there is a resistance decrease, i.e., the second landmark of positive value for acceleration sensor. Therefore, in the process of penetration the sensor may give two signals: the first negative value to locate bone reference, then depth control mechanism may be set at this point. The second positive value of the sensor will alarm the marrow entrance during further penetration, needle stop mechanism may be set then. Either of the two signals may be utilized for accurate penetration. The design may also utilize both of them, the safe penetration with depth control may be doubly guaranteed to meet unexpected and complicated habitus.

If the needle continues advancement after marrow entrance, the needle tip will reach the inner cortical bone, on the opposite side of the marrow, and there is a resistance increases again. This is the third landmark for over-penetration. A microprocessor may be programmed to analyze all possible landmarks, control electromagnet friction brake and give over-penetration alarm to guarantee the needle penetration in the safer and more reliable way.

In application of minimally invasive surgery, a restraint apparatus is designed to assist in the process of trocar insertion, especially the blind procedures of insufflation needle and prime trocar, with an add-on safety feature to greatly reduce the injury risk by two independent safety systems; the original safety feature on the trocar and the restraint in trocar or hand movements.

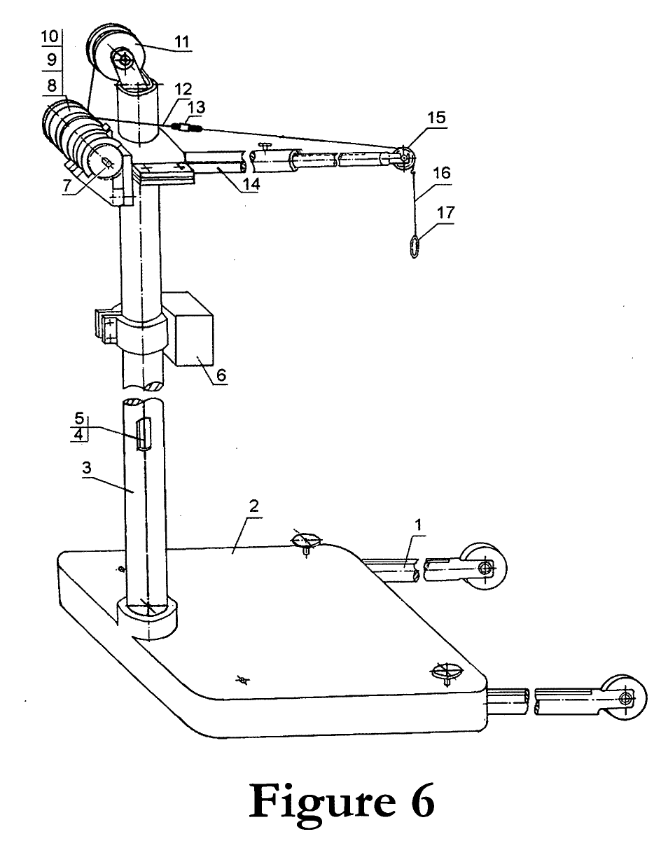

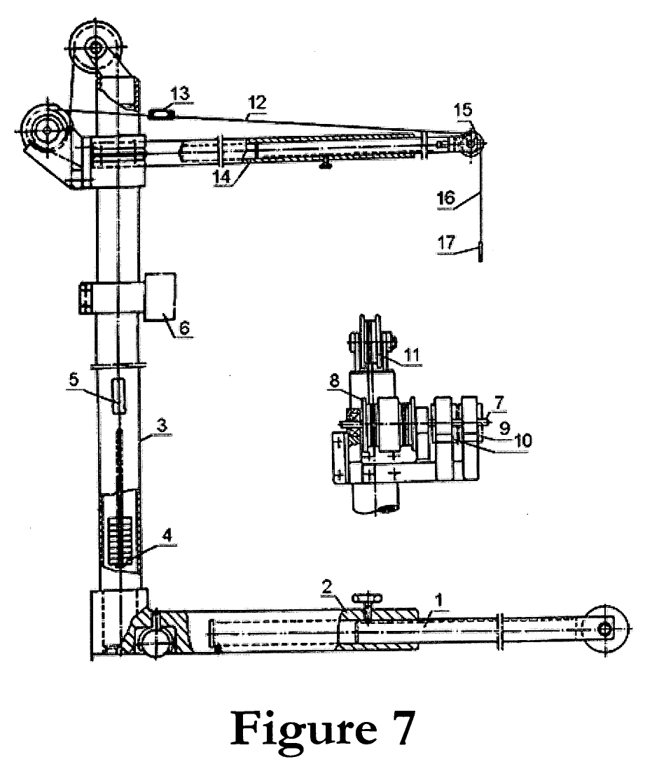

The framework of the restraint apparatus is illustrated in FIG. 6 and FIG. 7. A base 2 is provided at the bottom of the apparatus, with two extending rods 1 to strengthen stability in force withstanding circumstances. A vertical hollow bar 3 is seated on base 2. In the vicinity of the other end of bar 3, a horizontal arm 14 is mounted. Arm 14 consists of two pieces and the length may be adjusted. Near the fixture point of arm 14, a freely moving shaft 7 is fixed on bar 3 through two bearings. Shaft 7 has a plug-in bobbin with two winders 8 that may move together with shaft 7. Along the same axis of shaft 7 an angle encoder 9 and electric brake 10 are fixed on the bar with their moving parts plugging in shaft 7 in a parallel arrangement with bobbin 8.

A string 12 coming from one winder of bobbin 8 is pulled along arm 14 and rests at pulley 15. One tip of string 12 is fixed in winder of bobbin 8 and the other tip has a hook in connection with a strap 16. At the middle position of string 12 an acceleration sensor 13 is fixed. For a more accurate detection, the alternative position of acceleration sensor 13 is on the proximal portion of the trocar by mechanical clamp (not illustrated in FIG. 6 and FIG. 7). Theoretically the acceleration sensor may have both wired and wireless types, though the wired type has a lower cost for disposable use, such as commercially available MEMS (micro-electromechanical systems) one. Strap 16 is flexible and non-stretchable with the other tip forming an adjustable loop 17 to be secured to the proximal part of the trocar (possibly through a connection adapter) or practitioner's hands in an easy fastening and detachable connection.

Another string 5 coming from the other winder of bobbin 8 goes downwards inside hollow bar 3 via another pulley 11. The other tip of string 5 is connected with a balancing mass 4. Thus, string 5 and balancing mass 4 drive shaft 7 to rotate counter clockwise. Mass 4 is set to have the suitable weight that it may keep string 12 and strap 16 in proper tension and the practitioner may pull strap 16 easily without a feeling of burden, while strap 16 is secured on the proximal part of a trocar (possibly through a connection adapter) or practitioner's hand. For operations convenience, an operation panel and electrical case 6 is fixed at the middle position of the vertical bar 3.

Note that strings 5 and 12 may comprise wire, wound wire, plastic string, line, or rope, tape drive, chain, mechanical linkages or other types of linkage and are described here as string only for purposes of illustration. Preferably, a linkage material is provided which is not prone to stretching and may be readily sterilized. Similarly, although a rotary or angle encoder is illustrated herein, linear encoders and other types of position measurement devices may be used within the spirit and scope of the present invention.

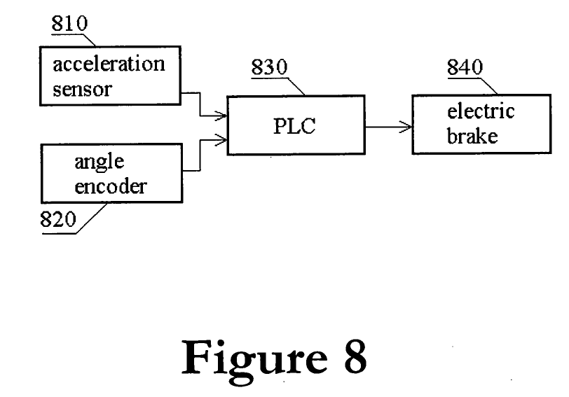

FIG. 8 is a block diagram illustrating the major components of the restraint mechanism of the present invention. Referring to FIGS. 6, 7, and 8, the basic working principle of the restraint apparatus is as follows. In the procedures of insufflation needle and trocar insertions, strap 16 line direction may be first adjusted to the axis of trocar insertion through apparatus position and/or arm length adjustment. Then strap loop 17 is secured on the proximal part of a trocar (possibly through a connection adapter) or the practitioner's hand (wrist or finger) and allows conventional insertion procedures. When the trocar tip just enters the cavity at earliest penetration stage, acceleration sensor 810 may give a feedback signal to the PLC (programmable logic controller) 830 in electrical case 6. The trocar penetration depth is approximately the further moving increment of attached strap 16 after cavity entrance, which may be detected and controlled by the precise angular position of rotary shaft 7.

The precise angular position of rotary shaft 7 may be measured by an angle encoder 820 with a very high accuracy as high as in the range of angular seconds and better. According to the acceleration sensor feedback for trocar tip entrance in the cavity and the angular position data of rotary shaft 7, PLC 830 may control a predetermined trocar depth in the cavity by means of actuating electric brake 840 (such as simplest miniature single-plate electromagnetic brake) to stop the shaft rotation at a predetermined angular position. Thus, the trocar movement or the practitioner's hand movement may be restrained so as to limit penetration depth of the trocar, no matter the safety feature on the trocar is deployed or not. In consideration of anatomic variations of body habitus from different patients, the further moving increment of strap 16 after cavity entrance may be conveniently set in various ways, automatically, manually or be repeated several times to give the best result in the cautious and safe way for each patient.

When the penetrating instrument sometimes goes through two unrelated different tissue layers in the penetration, there may be resistance change (acceleration). Usually the acceleration of this resistance change is much smaller than that of sudden lack of resistance upon cavity entrance and may not make false signal. For superior function, the PLC may be easily programmed to have different detection threshold values for trocars with different diameters or tip shapes (which may influence the acceleration value), as well as patient body habitus to reduce this small possibility. In fact, practitioners should know whether the instrument is in somewhere else other than the vicinity of cavity according to their essential anatomical knowledge. In case there comes a false signal from unrelated resistance change, the PLC may be just pressed by a foot switch or an assistant to reset. The strap restraint for the trocar movement or the practitioner's hand movement may be released and the penetration continues without any adverseness to safety.

The restraint apparatus may be a reusable one. Strap 16 in contact with the trocar or the practitioner's hand and acceleration sensor 13 when in the alternative position on the trocar may be disposable for single use.

Note that the restraint apparatus as described herein is by way of example only and illustrates the principle of the present invention and in no way should be construed as otherwise limiting the spirit and scope of the present invention. Other variations based upon the description of the present invention will be readily apparent to one of ordinary skill in the art. For example, the adjustment of the horizontal arm length and height may be varied. In addition, the base movement may be motor driven automatically instead of using a manual operation. The method to secure the trocar or practitioner's hand may be in the form of an arm support instead of a strap. In addition, the practitioner's front arm may rest on an arm support and move freely upwards, downwards, and horizontally. Upon the cavity entrance, the arm support may be immobilized in the downward direction so as to restrain the practitioner's hand movement.

While the preferred embodiment and various alternative embodiments of the invention have been disclosed and described in detail herein, it may be apparent to those skilled in the art that various changes in form and detail may be made therein without departing from the spirit and scope thereof.

I claim:

1. An apparatus for use with a penetrating instrument, comprising:
an acceleration sensor, coupled to the penetrating instrument, for measuring acceleration of the penetrating instrument and outputting an electronic signal;
a processing circuit, coupled to the acceleration sensor, for processing the electronic signal and outputting a signal indicative of penetration of the penetration instrument.

2. The apparatus of claim 1, further comprising an alarm coupled to the processing circuit, for generating an alarm when the penetrating instrument has reached a predetermined point of penetration.

3. The apparatus of claim 1, further comprising an actuating mechanism, coupled to the processing circuit, for controlling movement of the penetrating instrument.

4. The apparatus of claim 1, wherein the acceleration sensor measures or detects a sudden lack of resistance to penetration by the penetrating instrument.

5. The apparatus of claim 1, further comprising a pressure sensor for measuring at least one of a sign of negative pressure and a sign of a confirmatory pressure swing corresponding to the penetrating instrument penetration and outputting an electronic signal.

6. The apparatus of claim 5, wherein the pressure sensor outputs the electronic signal to the processing circuit, wherein the processing circuit combines the electronic signal from the pressure sensor with the electronic signal from the accelerometer to output the signal indicative of penetration of the penetration instrument.

7. The apparatus of claim 6, further comprising an alarm coupled to the processing circuit, for generating a first alarm for pressure and/or resistance change detection and then a second confirmatory alarm to ensure the correct needle position.

8. The apparatus of claim 5, wherein the pressure sensor outputs the electronic signal to the processing circuit, wherein the processing circuit processes the electronic signal from the pressure sensor and the electronic signal from the accelerometer to output a first signal in response to a sensed acceleration and a second signal in response to a sensed pressure.

9. The apparatus of claim 1, further comprising:
a depth measurement instrument, coupled to the penetration instrument, for controlling depth of penetration subsequent to the indication of penetration by the penetration instrument.

10. The apparatus of claim 3, wherein the actuating mechanism further comprises:
an encoder for measuring movement of the penetrating instrument, and
an electric brake, coupled to the penetrating instrument, for braking movement of the penetrating instrument once penetration has been detected.

11. A method of using a penetrating instrument, comprising the steps of:
inserting a penetrating instrument into one or more of a body, a body cavity, organ, and potential space,
measuring, with an acceleration sensor coupled to the penetrating instrument, acceleration of the penetrating instrument, and outputting an electronic signal,
processing, in a processing circuit coupled to the acceleration sensor, the electronic signal, and
outputting a signal indicative of penetration of the penetration instrument.

12. The method of claim 11, further comprising the step of generating an alarm with an alarm coupled to the processing circuit, when the penetrating instrument has reached a predetermined point of penetration.

13. The method of claim 11, further comprising the step of controlling movement of the penetrating instrument with an actuating mechanism coupled to the processing circuit.

14. The method of claim 11, wherein the acceleration sensor measures or detects a sudden lack of resistance to penetration by the penetrating instrument.

15. The method of claim 11, further comprising the steps of:
measuring with a pressure sensor, at least one of a sign of negative pressure and a sign of a confirmatory pressure swing corresponding to the penetrating instrument penetration, and
outputting an electronic signal corresponding to at least one of a sign of negative pressure and a sign of a confirmatory pressure swing corresponding to the penetrating instrument penetration.

16. The method of claim 15, further comprising the steps of:
outputting, from the pressure sensor, the electronic signal to the processing circuit, and
combining, in the processing circuit, the electronic signal from the pressure sensor with the electronic signal from the accelerometer to output the signal indicative of penetration of the penetration instrument.

17. The method of claim 16, further comprising the step of generating, with an alarm coupled to the processing circuit, a first alarm for pressure and/or resistance change detection and then a second confirmatory alarm to ensure the correct needle position.

18. The method of claim 15, further comprising the steps of:
outputting, from the pressure sensor, the electronic signal to the processing circuit,
processing, in the processing circuit, the electronic signal from the pressure sensor with the electronic signal from the accelerometer to output a first signal in response to a sensed acceleration, and
processing, in the processing circuit, the electronic signal from the pressure sensor to output a second signal in response to a sensed pressure.

19. The method of claim 11, further comprising the step of:
controlling depth penetration with a depth measurement instrument coupled to the penetration instrument, subsequent to the indication of penetration by the penetration instrument.

20. The method of claim 13, wherein the step of controlling movement of the penetrating instrument further comprises the steps of:
measuring movement of the penetrating instrument with an encoder, and
braking movement of the penetrating instrument with an electric brake coupled to the penetrating instrument, once penetration has been detected.
